(19) United States
(12) Patent Application Publication
(10) Pub. No.: US 2025/0027043 A1
(43) Pub. Date: Jan. 23, 2025

(54) IN VITRO EXPANSION METHOD FOR CULTIVATING MEMORY IMMUNE CELL

(71) Applicant: SHANGHAI NAICON BIOTECHNOLOGY CO., LTD., Pudong New Area (CN)

(72) Inventors: Yiyong WEI, Pudong New Area (CN); Rong YING, Pudong New Area (CN); Xiangbo CHEN, Pudong New Area (CN)

(21) Appl. No.: 18/899,858

(22) Filed: Sep. 27, 2024

Related U.S. Application Data
(63) Continuation of application No. PCT/CN2023/109713, filed on Jul. 28, 2023.

(30) Foreign Application Priority Data
Jul. 21, 2023 (CN) 202310901909.3

(51) Int. Cl. C12N 5/0783

(52) U.S. Cl. CPC C12N 5/0636; C12N 2501/2302; C12N 2501/2307; C12N 2501/2321

(57) ABSTRACT

An in vitro expansion method for cultivating memory immune cells is provided, and the method includes: 1) using a lymphocyte separation solution to separate immune cells from peripheral blood or single blood collection; 2) activating the immune cells obtained in step 1) with a first solution in complete culture medium; 3) after activating the immune cells, adding a second solution during culture and adjusting cell culture density at intervals; and 4) using different culture containers and comparing cell expansion folds and phenotypes to determine an optimal culture container and obtain the memory immune cells. On a basis of traditional immune cell culture medium, the disclosure optimizes a culture system (cytokine type and concentration, cell culture density and culture container) and can be applied to production as a preparation method of the memory immune cells, thus improving proportion and quantity of specific T cells.

### CROSS REFERENCE TO RELATED APPLICATIONS

This application is a Continuation of the International Application No. PCT/CN2023/109713 filed on Jul. 28, 2023, which claims priority to Chinese Patent Application No. 202310901909.3 on filed Jul. 21, 2023 under 35 U.S.C. § 119, the entire contents of all of which are hereby incorporated by reference.

### TECHNICAL FIELD

The disclosure belongs to the field of biotechnology and relates to immune cells, in particular to an in vitro expansion method for cultivating memory immune cells.

### BACKGROUND ART

Adoptive cellular immunotherapy, including in vitro expansion and transfusion of tumor-targeted T cells, such as tumor infiltrating lymphocytes (TIL) and recombinant T cells with conventional T cell receptors (TCRs) or chimeric antigen receptors (CARs), is gradually becoming second-line therapy for patients with advanced cancer. However, imbalance of metabolic activity of tumor cells results in immunosuppression of tumor microenvironment (TME), including soluble factors secreted by tumor or stromal cells, and suppressor immune cells such as myeloid-derived suppressor cells (MDSCs) and regulatory T cells (Tregs), which results in metabolic stress and failure of cellular immunotherapy.

Currently, most adoptive cellular immunotherapy focuses on targeting to tumor cells and introduces an antibody sequence by gene editing, ignoring functionality of T cells themselves.

Most of obtained T cells are terminally differentiated T cells (Teff), which are difficult to survive in the body for a long time.

### SUMMARY

In order to solve a problem that immune cells are easy to differentiate into terminal effector T cells during in vitro culture, an in vitro expansion method for cultivating memory immune cells is proposed in the disclosure, which uses peripheral blood or single blood collection as a cell source, separates lymphocytes from the cell source, and then further induces, expands and cultivates memory T cells in complete medium of immune cells. A total culture duration is 10 to 15 days, during which fresh complete medium is continuously added in light of cell density and culture density is adjusted.

In order to achieve the above object, the disclosure adopts following technical schemes.

An in vitro expansion method for cultivating memory immune cells is provided in the disclosure, which includes following steps:
1) using a lymphocyte separation solution to separate immune cells from peripheral blood or single blood collection;
2) activating the immune cells obtained in step 1) with a first solution in complete culture medium;
3) after activating the immune cells, adding a second solution during culture and adjusting cell culture density at intervals; and
4) using different culture containers and comparing cell expansion folds and phenotypes to determine an optimal culture container and obtain the memory immune cells.

As a preferred embodiment of the present disclosure, in step 2), the first solution contains following components: first immune cell culture medium with 10 to 300 IU/ml of IL-2, 5 to 500 ng/ml of IL-21 and 20 to 250 IU/ml of IL-7, and activated magnetic beads. If there is too much IL-2, cell expansion may be too fast, and a proportion of the memory T cells may decrease; and if there is too little IL-2, it may not function in cell expansion. If there are too much IL-7 and IL-21, it may stimulate cytokine storm inside T cells and cause the T cells to apoptosis quickly; and if there are too little IL-7 and IL-21, they may not function in cell differentiation.

As a preferred embodiment of the present disclosure, the first immune cell culture medium is one of ImmunoCult-XF, TexMACS or X-VIVO.

As a preferred embodiment of the present disclosure, in step 3), the second solution contains following components: second immune cell culture medium with 50 to 500 IU/ml of IL-2, 10 to 1000 ng/ml of IL-21, 50 to 300 IU/ml of IL-7, and a serum substitute. If there is too much IL-2, cell expansion may be too fast, and a proportion of the memory T cells may decrease; and if there is too little IL-2, it may not function in cell expansion. If there are too much IL-7 and IL-21, it may stimulate cytokine storm inside T cells and cause the T cells to apoptosis quickly; and if there are too little IL-7 and IL-21, they may not function in cell differentiation.

As a preferred embodiment of the disclosure, contents of IL-2, IL-7 and IL-21 in the second solution are higher than those in the first solution.

As a preferred embodiment of the present disclosure, the second immune cell culture medium is one of ImmunoCult-XF, TexMACS or X-VIVO.

Alternatively, the first immune cell culture medium may be the same as or different from the second immune cell culture medium.

As a preferred embodiment of the disclosure, the first solution in step 2) activates immune cells on a day DO, and cell density is adjusted to 1 to 4E⁶/mL.

As a preferred embodiment of the present disclosure, a culture duration in step 3) is up to a day D12, 2 to 3 times the volume of the second solution is added on a day D2, and the cell density is adjusted to 5E⁴ to 1E⁶/mL on a day D4, to 6E⁴ to 1.2E⁶/mL on a day D6, to 7E⁴ to 1.4E⁶/mL on a day D8, and to 8E⁴ to 1.6E⁶/mL on a day D10.

As a preferred embodiment of the present disclosure, the culture containers in step 4) include a G-Rex culture container, an Origen culture bag, a Takara culture bag and a traditional culture flask.

As a preferred embodiment of the disclosure, step 1) specifically includes: taking a 50 mL centrifugal tube and adding a separation solution with a same volume as a blood sample; slowly adding the blood sample to the separation solution with a pipette, and taking 500 to 1000 g of this mixture for centrifuging for 20 to 30 min; after centrifugation, sucking a second layer of annular milky white lymphocytes into a new centrifuge tube and adding 10 ml of PBS (phosphate buffer saline) to mix with the cells evenly; taking 250 g of this mixture, centrifuging for 10 min and discarding supernatant to obtain the immune cells.

Compared with related art, the disclosure provides following beneficial effects.

1) On a basis of traditional immune cell culture medium, the disclosure optimizes a culture system (cytokine type and concentration, cell culture density and culture container) and can be applied to production as a preparation method of the memory immune cells, thus improving proportion and quantity of specific T cells.

2) In this disclosure, peripheral blood or single blood collection is used as a cell source, lymphocytes are separated from the cell source, and then memory T cells are further induced, expanded and cultivated in the complete medium of the immune cells.

3) This disclosure has broken mindset of those skilled in the art. Those skilled in the art generally think that expansion fold, viability and phenotype cannot be compatible, and in cultivating T cells, the culture duration tends to be set to 14 days or even more than 16 days. However, in this disclosure, when the culture container is set to be the Origen™ culture bag or the Takara™ culture bag, activation is performed with the first solution firstly. Then the second solution was used for expansion culture until a day D12, and final expansion fold, viability and phenotype all showed good performance, in which the cell viability was about 80%, cell expansion reached about 120 times, and the proportion of the memory T cells reached more than 80% at the day D12, even more than 90% for the Takara culture bag.

### BRIEF DESCRIPTION OF THE DRAWINGS

FIG. 1 is a schematic view of cell expansion folds of different groups in Example 1 of the present disclosure;

FIG. 2 is a schematic view of cell viability of different groups in Example 1 of the present disclosure; and

FIG. 3 is a schematic view of cell phenotype of different groups in Example 1 of the present disclosure.

### DETAILED DESCRIPTION

In order to make technical schemes of the disclosure more clear, the disclosure will be further explained in detail in the following in combination with drawings and specific examples.

An in vitro expansion method for cultivating memory immune cells is provided in an embodiment of the disclosure, which uses peripheral blood or single blood collection as a cell source, separates lymphocytes from the cell source, and then further induces, expands and cultivates memory T cells in complete medium of immune cells. A total culture duration is 10 to 15 days, during which fresh complete medium is continuously added in light of cell density and culture density is adjusted.

### EXAMPLE

The in vitro expansion method for cultivating memory immune cells disclosed in this embodiment includes following steps.

I. Alymphocyte separation solution was used to separate immune cells from peripheral blood or single blood collection, in which

(1) the adopted lymphocyte separation solution is GE Ficoll-Paque (article number: 17-1440-02) of GE Company;

(2) a 50 mL centrifugal tube was taken and a separation solution with a same volume as a blood sample was added;

(3) the blood sample was slowly added to the separation solution with a pipette, 500 to 1000 g of this mixture was taken for centrifuging for 20 to 30 min;

(4) after centrifugation, a second layer of annular milky white lymphocytes was sucked into a new centrifuge tube and 10 ml of PBS (phosphate buffer saline) was added to mix with the cells evenly; and

(5) 250 g of this mixture was taken for centrifuging for 10 min and supernatant is discarded to obtain the immune cells.

II. The immune cells was activated with activated magnetic beads, in which

1) the cells were resuspended with a first solution, and cell density was adjusted to 1 to 4E⁶/mL, with a temperature of 37° C. and 5% of CO₂; and

2) components of the first solution are as follows: first immune cell culture medium is ImmunoCult-XF or TexMACS or X-VIVO (all of three immune cell cultures can be used, and in this example ImmunoCult-XF with 100 IU/ml of interleukin IL-2, 20 ng/ml of interleukin IL-21 and 50 IU/ml of interleukin IL-7 and activated magnetic beads (Miltenyi, 130-111-160) were used).

III. After the immune cells were activated, cell culture density was continuously adjusted in which

The immune cells were activated by the first solution on a day DO, and then continuously cultured until a day D12.

1) 2 to 3 times the volume of the second solution was added on D2,

2) the cell density was adjusted to 5E⁵/mL on D4,

3) the cell density was adjusted to 6E⁵/mL on D6,

4) the cell density was adjusted to 7E⁵/mL on D8, and

5) the cell density was adjusted to 8E⁵/mL on D10.

Components of a second solution were as follows: second immune cell culture medium is ImmunoCult-XF or TexMACS or X-VIVO (all of three immune cell cultures can be used, and ImmunoCult-X was adopted in this example), with 50 IU/ml of IL-2, 30 ng/ml of IL-21, 100 IU/ml of IL-7, and 2% of a serum substitute (SR).

IV. Different culture containers were used, cell expansion folds and phenotypes were compared in which

1) different container groups are as follows:

Group 1 is a G-Rex culture container, Group 2 is an Origen culture bag, Group 3 is a Takara culture bag, and Group 4 is a traditional culture flask.

Results are as follows.

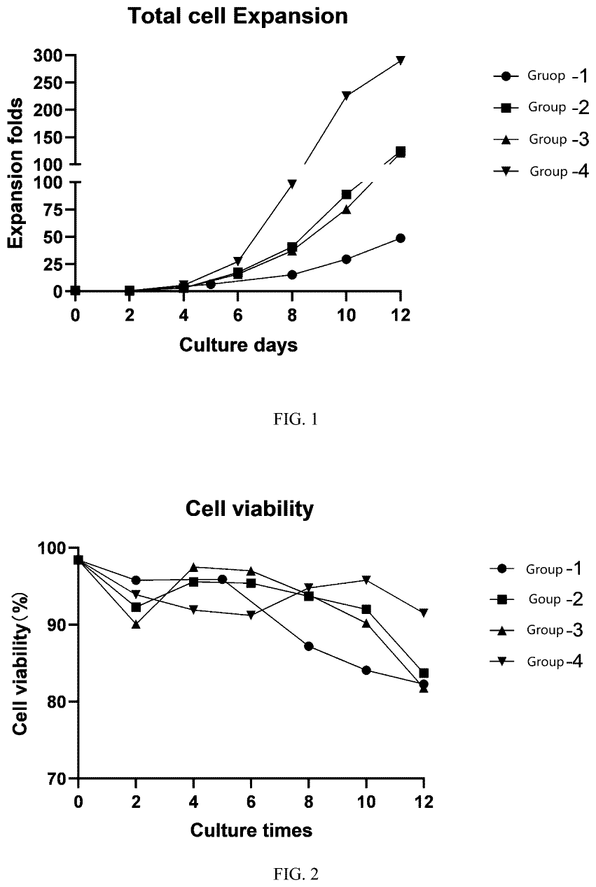

Refer to FIGS. 1 and 2, expansion folds and viability are shown.

As shown in FIG. 1, it can be seen from final expansion folds of cells on D12, expansion folds of Group 4 is highest, which can reach about 300; followed by two culture bag groups (Groups 2 and 3), their expansion folds are close and are about 120; the worst is Group 1, which was finally expanded at a fold of less than 50.

As shown in FIG. 2, the cell viability is shown as follows: Group 4 is relatively stable, and finally with viability of about 90%; and the other three groups show a downward trend after a day D6, and is with final cell viability of about 80%.

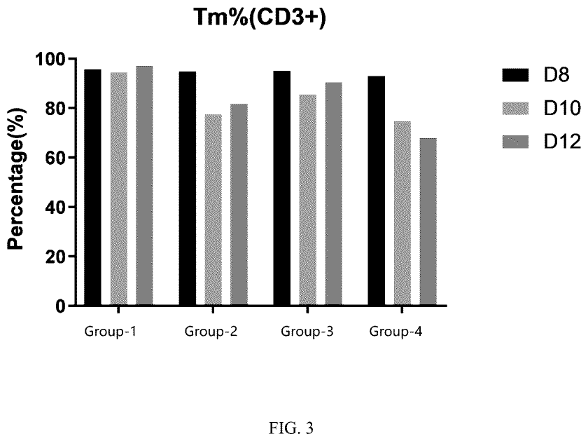

As shown in FIG. 3, cell phenotypes were measured on days D8/10/12, respectively. Group 1 is relatively stable and is with more than 95% of memory T cells (TM). Group 4 shows a downward trend and is with a final proportion of memory T cells of about 70%. Phenotypes of Groups 2 and 3 decrease on the day D10 and rise on the day D12, and Group 3 (92.5%) is better than Group 2 (80%).

To sum up, as a preferred example, when the culture container is the Origen culture bag or the Takara culture bag, the first solution is firstly used for activation, and then the second solution is used for expansion culture until the day D12, and the final expansion fold, viability and phenotype all have good performance.

TABLE 1
Data of Examples and Comparative Examples

| | | | | | | | | | | | | Maximum |
|---|---|---|---|---|---|---|---|---|---|---|---|---|
| | | | | | | | | | | | | proportion |
| | | | | | | | Cell | Cell | Cell | Cell | | of memory |
| | | | | | | | density | density | density | density | | T cells |
| | First solution | | | Second solution | | | on day | on day | on day | on day | Culture | on days |
| | IL-2 | IL-21 | IL-7 | IL-2 | IL-21 | IL-7 | D 4 | D 6 | D 8 | D 10 | bag | D 8/10/12 |
| Exam- | 100 | 20 | 50 | 50 | 30 | 100 | 5E⁵/mL | 6E⁵/mL | 7E⁵/mL | 8E⁵/mL | Takara | 92.5% |
| ple 1 | | | | | | | | | | | culture | |
| | | | | | | | | | | | bag | |
| Exam- | 10 | 5 | 20 | 50 | 10 | 50 | As with | As with | As with | As with | Takara | 86.7% |
| ple 2 | | | | | | | Exam- | Exam- | Exam- | Exam- | culture | |
| | | | | | | | ple 1 | ple 1 | ple 1 | ple 1 | bag | |
| Exam- | 300 | 500 | 250 | 500 | 1000 | 300 | As with | As with | As with | As with | Takara | 80.2% |
| ple 3 | | | | | | | Exam- | Exam- | Exam- | Exam- | culture | |
| | | | | | | | ple 1 | ple 1 | ple 1 | ple 1 | bag | |
| Exam- | 150 | 250 | 150 | 250 | 500 | 150 | As with | As with | As with | As with | Takara | 89.3% |
| ple 4 | | | | | | | Exam- | Exam- | Exam- | Exam- | culture | |
| | | | | | | | ple 1 | ple 1 | ple 1 | ple 1 | bag | |
| Exam- | As with | As with | As with | As with | As with | As with | 1E⁶/mL | 1.2E⁶/mL | 1.4E⁶/mL | 1.6E⁶/mL | Takara | 83.5% |
| ple 5 | Exam- | Exam- | Exam- | Exam- | Exam- | Exam- | | | | | culture | |
| | ple 1 | ple 1 | ple 1 | ple 1 | ple 1 | ple 1 | | | | | bag | |
| Exam- | As with | As with | As with | As with | As with | As with | 7E⁵/mL | 1E⁶/mL | 1.2E⁶/mL | 1.4E⁶/mL | Takara | 85.8% |
| ple 6 | Exam- | Exam- | Exam- | Exam- | Exam- | Exam- | | | | | culture | |
| | ple 1 | ple 1 | ple 1 | ple 1 | ple 1 | ple 1 | | | | | bag | |
| Exam- | As with | As with | As with | As with | As with | As with | 5E⁴/mL | 6E⁴/mL | 7E⁴/mL | 8E⁴/mL | Takara | 81.5% |
| ple 7 | Exam- | Exam- | Exam- | Exam- | Exam- | Exam- | | | | | culture | |
| | ple 1 | ple 1 | ple 1 | ple 1 | ple 1 | ple 1 | | | | | bag | |
| Compar- | 400 | 600 | 350 | 600 | 1100 | 400 | As with | As with | As with | As with | Takara | 63.8% |
| ative | | | | | | | Exam- | Exam- | Exam- | Exam- | culture | |
| Exam- | | | | | | | ple 1 | ple 1 | ple 1 | ple 1 | bag | |
| ple 1 | | | | | | | | | | | | |
| Compar- | As with | As with | As with | As with | As with | As with | 1.1E⁶/mL | 1.3E⁶/mL | 1.5E⁶/mL | 1.7E⁶/mL | Takara | 78.2% |
| ative | Exam- | Exam- | Exam- | Exam- | Exam- | Exam- | | | | | culture | |
| Exam- | ple 1 | ple 1 | ple 1 | ple 1 | ple 1 | ple 1 | | | | | bag | |
| ple 2 | | | | | | | | | | | | |
| Compar- | 100 | 20 | 50 | 100 | 20 | 50 | As with | As with | As with | As with | Takara | 77% |
| ative | | | | | | | Exam- | Exam- | Exam- | Exam- | culture | |
| Exam- | | | | | | | ple 1 | ple 1 | ple 1 | ple 1 | bag | |
| ple 3 | | | | | | | | | | | | |

### Examples 2 to 7

In all of Examples 2 to 7, the first solution was used for activation and then the second solution was used for expansion culture until the day D12.

Concentrations of respective components and cell concentrations are shown in Table 1, with the remaining the same as in Example 1.

Finally, the cell viability is about 80%, the cell expansion was at a fold of about 120, and the proportion of memory T cells reached more than 80% on the day D12.

### Comparative Examples 1 to 3

In all of Comparative Examples 1 to 3, the first solution was used for activation and then the second solution was used for expansion culture until the day D12.

Concentrations of respective components and cell concentrations are shown in Table 1, with the remaining the same as in Example 1.

Finally, in Comparative Example 1, concentration of cytokines is too high, which may results in premature apoptosis and exhaustion of T cells and differentiation to terminal T cells, and thus no memory function on the day D12 day, and proportion of finally collected memory T cells is greatly reduced, only 63.8%.

In Comparative Example 2, density of T cells exceeds a critical range. If the cell density is too high, the cells activate each other, and the cells may expand rapidly at an early stage and cell expansion slows down at a later stage, resulting in phenomenon of cell apoptosis. On the day D12, the proportion of memory T cells is less than 80% (78.2%).

In Comparative Example 3, contents of IL-2, IL-7 and IL-21 in the second solution are equal to those in the first solution. Finally, the proportion of memory T cells is less than 80% (77%).

The above described embodiments only express several implementations of the present disclosure, and their descriptions are more specific and detailed, but they cannot be constructed as limiting a scope of the present invention. It should be noted that, several modifications and improvements can be made by those of ordinary skill in the art without departing from the concept of the present invention, which belong to the protection scope of the present invention. Therefore, the protection scope of this disclosure shall be subjected to appended claims.

1. An in vitro expansion method for cultivating memory immune cells, comprising following steps:
1) using a lymphocyte separation solution to separate immune cells from peripheral blood or single blood collection;
2) activating the immune cells obtained in step 1) with a first solution in complete culture medium;
3) after activating the immune cells, adding a second solution during culture and adjusting cell culture density at intervals; and
4) using different culture containers and comparing cell expansion folds and phenotypes to determine an optimal culture container and obtain the memory immune cells.

2. The in vitro expansion method for cultivating memory immune cells according to claim 1, wherein in step 2), the first solution contains following components: first immune cell culture medium with 10 to 300 IU/ml of IL-2, 5 to 500 ng/ml of IL-21 and 20 to 250 IU/ml of IL-7, and activated magnetic beads.

3. The in vitro expansion method for cultivating memory immune cells according to claim 2, wherein the first immune cell culture medium is one of ImmunoCult™-XF, TexMACS™ or X-VIVO™.

4. The in vitro expansion method for cultivating memory immune cells according to claim 1, wherein in step 3), the second solution contains following components: second immune cell culture medium with 50 to 500 IU/ml of IL-2, 10 to 1000 ng/ml of IL-21, 50 to 300 IU/ml of IL-7, and a serum substitute.

5. The in vitro expansion method for cultivating memory immune cells according to claim 4, wherein the second immune cell culture medium is one of ImmunoCult™-XF, TexMACS™ or X-VIVO™.

6. The in vitro expansion method for cultivating memory immune cells according to claim 1, wherein the first solution in step 2) activates immune cells on a day DO, and cell density is adjusted to 1 to 4E⁶/mL.

7. The in vitro expansion method for cultivating memory immune cells according to claim 6, wherein a culture duration in step 3) is up to a day D12, 2 to 3 times the volume of the second solution is added on a day D2, and the cell density is adjusted to 5E⁴ to 1E⁶/mL on D4, to 6E⁴ to 1.2E⁶/mL on a day D6, to 7E⁴ to 1.4E⁶/mL on a day D8, and to 8E⁴ to 1.6E⁶/mL on a day D10.

8. The in vitro expansion method for cultivating memory immune cells according to claim 1, wherein the culture containers in step 4) comprise a G-Rex culture container, an Origen culture bag, a Takara culture bag and a traditional culture flask.

9. The in vitro expansion method for cultivating memory immune cells according to claim 1, wherein step 1) specifically comprises: taking a 50 mL centrifugal tube and adding a separation solution with a same volume as a blood sample; slowly adding the blood sample to the separation solution with a pipette, and taking 500 to 1000 g of this mixture for centrifuging for 20 to 30 min; after centrifugation, sucking a second layer of annular milky white lymphocytes into a new centrifuge tube and adding 10 ml of PBS to mix with the cells evenly; taking 250 g of this mixture, centrifuging for 10 min and discarding supernatant to obtain the immune cells.

10. The in vitro expansion method for cultivating memory immune cells according to claim 7, wherein the culture containers in step 4) are Origen culture bags or Takara culture bags.
